(19) United States
(12) Patent Application Publication
(10) Pub. No.: US 2003/0207249 A1
(43) Pub. Date: Nov. 6, 2003

(54) Connection of cells to substrates using association pairs

(76) Inventors: Oren E. Beske, Sunnyvale, CA (US); Simon Goldbard, San Jose, CA (US)

Correspondence Address: KOLISCH HARTWELL, P.C., 520 S.W. YAMHILL STREET, SUITE 200, PORTLAND, OR, 97204, US

(21) Appl. No.: 10/407,630

(22) Filed: Apr. 4, 2003

Related U.S. Application Data
(63) Continuation-in-part of application No. 09/694,077, filed on Oct. 19, 2000, now abandoned.
(63) Continuation-in-part of application No. 10/120,900, filed on Apr. 10, 2002.
(60) Provisional application No. 60/370,313, filed on Apr. 4, 2002.
(60) Provisional application No. 60/129,664, filed on Apr. 15, 1999.
(60) Provisional application No. 60/170,947, filed on Dec. 15, 1999.

(30) Foreign Application Priority Data
Oct. 18, 2001 (WO) PCT/US01/51413

(51) Int. Cl. C12Q 1/00; G01N 33/53; G01N 33/567

(52) U.S. Cl. 435/4; 435/7.2; 435/7.5

(57) ABSTRACT

The invention provides systems, including methods, apparatus, compositions, and kits, for connection of cells or cell structures to substrates, such as coded particles or microplates, utilizing association pairs that are chemically reactive or specifically bind one another.

### CROSS-REFERENCE TO PRIORITY APPLICATIONS

This application is a continuation-in-part of the following U.S. patent applications Ser. No. 09/694,077, filed Oct. 19, 2000; and Ser. No. 10/120,900, filed Apr. 10, 2002. This application also claims the benefit under 35 U.S.C. § 119(e) of U.S. Provisional Patent Application Serial No. 60/370,313, filed Apr. 4, 2002.

U.S. patent application Ser. No. 09/694,077 is a continuation-in-part of U.S. patent application Ser. No. 09/549,970, filed Apr. 14, 2000, which in turn claims the benefit under 35 U.S.C. § 119(e) of the following U.S. provisional patent applications: Serial No. 60/129,664, filed Apr. 15, 1999; and Serial No. 60/170,947, filed Dec. 15, 1999.

U.S. patent application Ser. No. 10/120,900 claims the benefit under 35 U.S.C. § 120 of PCT Application Serial No. PCT/US01/51413, filed Oct. 18, 2001, and published as Publication No. WO 02/37944 on May 16, 2002, which in turn claims priority from the following U.S. provisional patent applications: Serial No. 60/241,714, filed Oct. 18, 2000; Serial No. 60/259,416, filed Dec. 28, 2000; Serial No. 60/293,863, filed May 24, 2001; Serial No. 60/299,267, filed Jun. 18, 2001; Serial No. 60/299,810, filed Jun. 20, 2001; Serial No. 60/307,649, filed Jul. 24, 2001; Serial No. 60/307,650, filed Jul. 24, 2001; Serial No. 60/310,540, filed Aug. 6, 2001; Serial No. 60/317,409, filed Sep. 4, 2001; Serial No. 60/318,156, filed Sep. 7, 2001; and Serial No. 60/328,614, filed Oct. 10, 2001.

The above-identified U.S., PCT, and provisional patent applications are all incorporated herein by reference in their entirety for all purposes.

### CROSS-REFERENCE TO RELATED APPLICATIONS

This application incorporates by reference in their entirety for all purposes the following U.S. patent application Ser. No. 09/694,011, filed Oct. 19, 2000; Ser. No. 10/119,814, filed Apr. 9, 2002; Ser. No. 10/186,219, filed Jun. 27, 2002; Ser. No. 10/238,914, filed Sep. 9, 2002; Ser. No. 10/273,605, filed Oct. 18, 2002; Ser. No. 10/282,904, filed Oct. 28, 2002; Ser. No. 10/282,940, filed Oct. 28, 2002; Ser. No. 10/382,796, filed Mar. 5, 2003; Ser. No. 10/382,797, filed March 5; and Ser. No. 10/382,818, filed Mar. 5, 2003.

This application also incorporates by reference in their entirety for all purposes the following U.S. provisional patent applications: Serial No. 60/383,091, filed May 23, 2002; Serial No. 60/383,092, filed May 23, 2002; Serial No. 60/413,407, filed Sep. 24, 2002; Serial No. 60/413,675, filed Sep. 24, 2002; Serial No. 60/421,280, filed Oct. 25, 2002; and Serial No. 60/426,633, filed Nov. 14, 2002.

This application also incorporates by reference in their entirety for all purposes the following PCT patent application: Serial No. PCT/US00/10181, filed Apr. 14, 2000, and published as Publication No. WO 00/63419 on Oct. 26, 2000; Serial No. PCT/US02/33350, filed Oct. 18, 2002; and Serial No. PCT/US02/34699, filed Oct. 28,2002.

### CROSS-REFERENCE TO ADDITIONAL MATERIALS

This application also incorporates by reference in their entirety for all purposes the following U.S. Pat. No. 3,772,099, issued Nov. 13, 1973; No. 3,897,284, issued Jul. 29, 1975; No. 3,964,294, issued Jun. 22, 1976; No. 3,966,599, issued Jun. 29, 1976; No. 3,980,561, issued Sep. 14, 1976; No. 4,053,433, issued Oct. 11, 1977; No. 4,087,327, issued May 2, 1978; No. 4,131,064, issued Dec. 26, 1978; No. 4,197,104, issued Apr. 8, 1980; No. 4,329,393, issued May 11, 1982; No. 4,343,904, issued Aug. 10, 1982; No. 4,363,965, issued Dec. 14, 1982; No. 4,390,452, issued Jun. 28, 1983; No. 4,469,623, issued Sep. 4, 1984; No. 4,634,675, issued Jan. 6, 1987; No. 4,640,035, issued Feb. 3, 1987; No. 4,649,114, issued Mar. 10, 1987; No. 4,652,395, issued Mar. 24, 1987; No. 4,727,040, issued Feb. 23, 1988; No. 4,833,083, issued May 23, 1989; No. 4,888,294, issued Dec. 19, 1989; No. 4,906,577, issued Mar. 6, 1990; No. 4,921,792, issued May 1, 1990; No. 4,963,490, issued Oct. 16, 1990; No. 4,982,739, issued Jan. 8, 1991; No. 5,019,512, issued May 28, 1991; No. 5,079,161, issued Jan. 7, 1992; No. 5,081,036, issued Jan. 14, 1992; No. 5,096,814, issued Mar. 17, 1992; No. 5,100,783, issued Mar. 31, 1992; No. 5,100,799, issued Mar. 31, 1992; No. 5,114,853, issued May 19, 1992; No. 5,126,269, issued Jun. 30, 1992; No. 5,233,369, issued Aug. 3, 1993; No. 5,409,839, issued Apr. 25, 1995; No. 5,451,505, issued Sep. 19, 1995; No. 5,486,855, issued Jan. 23, 1996; No. 5,571,410, issued Nov. 5, 1996; No. 5,708,153, issued Jan. 13, 1998; No. 5,741,462, issued Apr. 21, 1998; No. 5,760,394, issued Jun. 2, 1998; No. 5,770,455, filed Jun. 23, 1998; No. 5,780,258, issued Jul. 14, 1998; issued Jun. 23, 1998; No. 5,817,751, issued Oct. 6, 1998; No. 5,840,485, issued Nov. 24, 1998; No. 5,961,923, issued Oct. 5, 1999; No.5,981,180, issued Nov. 9, 1999; No. 5,989,835, issued Nov. 23, 1999; No. 5,990,479, issued Nov. 23, 1999; No. 6,025,200, issued Feb. 15, 2000; No. 6,100,026, issued Aug. 8, 2000; and No. 6,103,479, issued Aug. 15, 2000.

This application also incorporates by reference in their entirety for all purposes the following PCT Patent Applications: Serial No. PCT/IL97/00105, filed Mar. 20, 1997; Serial No. PCT/US98/21562, filed Oct. 14, 1998; Serial No. PCT/US98/22785, filed Oct. 27, 1998; Serial No. PCT/US99/00918, filed Jan. 15, 1999; Serial No. PCT/US99/01315, filed Jan. 22, 1999; Serial No. PCT/GB99/00457, filed Feb. 15, 1999; Serial No. PCT/US99/14387, filed Jun. 24, 1999; Serial No. PCT/GB99/02108, filed Jul. 2, 1999; Serial No. PCT/SE99/01836, filed Oct. 12, 1999; Serial No. PCT/US99/31022, filed Dec. 28, 1999; Serial No. PCT/US00/25457, filed Sep. 18, 2000; Serial No. PCT/US00/27121, filed Oct. 2, 2000; and Serial No. PCT/US00/41049, filed Oct. 2, 2000.

### FIELD OF THE INVENTION

The invention relates to connection of cells or subcellular structures to substrates. More particularly, the invention relates to connection of cells or subcellular structures to substrates, such as coded particles or microplates, by utilizing association pairs that are chemically reactive or that specifically bind to one another.

### BACKGROUND OF THE INVENTION

Cell analyses may benefit from, or be dependent on, attachment of cells to substrates. In these analyses, the substrate may perform at least several roles. First, the substrate may contribute to the growth and health of the cells, since many cells are unable to divide or even survive in the absence of an appropriate substrate. Second, the substrate may permit the cells to be organized and manipulated as a group by their attachment to the substrate; thus, the substrate may facilitate controllable and reversible exposure of the cells to various analytical reagents, media changes, and/or washing steps. Third, the substrate may permit the cells to be identified based on indicia of the substrate (such as an identifying code on a particle), position of the substrate, and/or position of the cells on the substrate; thus, the substrate may allow the cells to be organized and analyzed as an array. Fourth, the substrate may function as an examination site, the surface of which localizes the cells and promotes analysis by methods that may benefit from precise localization of sample, such as optical analysis.

The various functions of the substrate may be enabled more effectively when the substrate forms a sufficiently stable association with the cells. For example, if the cells adhere poorly to the substrate (or are substantially nonadherent, as with cells grown in suspension), processes as simple as a change in the growth medium may remove cells. Moreover, such problems may be magnified if the cells are exposed to a series of different solutions, as in a ligand-binding assay.

In some assays, substantial cell loss during growth and experimental manipulation may be acceptable, for example, when only a small fraction of cells on a substrate are analyzed. However, improved efficiency and throughput in drug screens may require a more efficient use of cells, with less cell loss during experimental analysis. Cell loss may become even more problematic in high-throughput screens that use positional or nonpositional cell arrays carried on a nonpartitioned planar substrate or on coded particles, respectively. In these screens, cells may detach and then re-attach at incorrect positions on the planar substrate or to different coded particles, thus increasing background noise and the number of cells that may need to be analyzed.

Increasing the affinity of cell-substrate interactions may improve the efficiency of assays, but in some assay systems a nonspecific increase in affinity may not be optimal. Such assay systems may employ a mixed population of cells, with only a fraction of cells being of interest. For example, the mixed population may be naturally occurring, such as a blood sample, a dispersion of cells from tissue, or a fluid aspirate, among others. Alternatively, the mixed population may result from modification of only a subset of a cell population, such as through transfection of a nucleic acid. Transfection generally introduces the nucleic acid into a fraction of the population, sometimes much less than 1% of cells in the population. Whatever the origin of the mixed population, cells not of interest in the population may interfere with analysis of the cells of interest. These interfering cells occupy space on the substrate, may contribute to background, may consume reagents, and may slow analysis of the cells of interest. Therefore, systems are needed that improve the affinity and/or selectivity of interactions between cells and substrates.

Assays with disrupted cells also may use a substrate to promote analysis. For example, ligand-binding assays may employ membrane fractions attached to substrates for “membrane assays,” instead of attaching whole cells (or purified receptors). In this case, the membrane fractions may carry receptors of interest as ligand-binding targets. The membrane fractions may be attached to the substrate, for example, the surface of a microplate well, using relatively nonspecific interactions, similar to those used to attach whole cells. Such membrane assays may be popular for high-throughput screens, because the results may provide a reliable, direct measure of ligand-receptor interactions. These membrane assays may be performed in various formats and measured using various readout systems (for example, scintillation proximity assays (SPA), fluorescent binding assays, etc.). Membrane assays may have at least several advantages over whole-cell assays: (1) membrane fractions may be prepared in bulk and easily stored frozen, (2) membrane fractions may produce a high concentration of receptor per area, when attached to a substrate, and (3) membrane fractions may retain most of the ligand-binding properties of the whole cells from which they were isolated, but may show less intrinsic variability than the whole cells.

Despite their popularity, such membrane assays suffer from some of the same problems faced by whole-cell assays. For example, membranes may tend to detach from the substrate, reducing signal and increasing variability in the assay. Moreover, mixed cell populations may contribute a majority of membranes that do not include the receptor of interest, reducing signal while increasing background. In addition, a mixture of different membranes carrying different targets cannot be resolved readily into discrete identifiable units during analysis. Accordingly, membranes from different cell types that express different receptors of interest may require separate compartments for use in a binding analysis, unless the membranes can be connected to different substrates disposed within the same compartment. Therefore, systems also are needed that improve the affinity and/or selectivity of connection between substrates and subcellular structures, such as membranes.

### SUMMARY OF THE INVENTION

The invention provides systems, including methods, apparatus, compositions, and kits, for connection of cells or subcellular structures to substrates, such as those defined by coded carriers or microplates, by utilizing associating pairs that are chemically reactive or that specifically bind to one another.

### BRIEF DESCRIPTION OF THE DRAWINGS

FIG. 1 is a schematic view of a method of connecting cells to a coded particle with an association pair, in accordance with aspects of the invention.

FIG. 2 is an enlarged view of the coded particle and cells of FIG. 1, with the cells connected to the coded particle using a specific binding pair as the association pair, in accordance with aspects of the invention.

FIG. 3 is an enlarged view of an alternative embodiment of the coded particle and cells of FIG. 1, with the cells connected covalently to the coded particle by covalent bonds produced by chemical reaction of members of the association pair, in accordance with aspects of the invention.

FIG. 4 is a schematic view of a method of covalently linking a specific binding member to cells using association members that are chemically reactive, in accordance with aspects of the invention.

FIG. 5 is a schematic view of a method of connecting cells to coded particles by metabolically labeling the cells with a chemically reactive member of an association pair, in accordance with aspects of the invention.

FIG. 6 is a schematic view of a method of forming the covalent bonds between the cells and coded particle of FIG. 3, in accordance with aspects of the invention.

FIG. 7 is a schematic sectional view of cells connected to a microplate well using an association pair, in accordance with aspects of the invention.

FIG. 8 is a schematic sectional view of cells connected to sub-wells within a microplate well using an association pair, in accordance with aspects of the invention.

FIG. 9 is a schematic view of a method of nonselectively connecting transfected and nontransfected cells to coded particles.

FIG. 10 is a schematic view of a method of selectively and/or more stably connecting transfected cells to coded particles, in accordance with aspects of the invention.

FIG. 11 is a schematic view of a method of using coded particles to screen compounds for their ability to compete with labeled ligands for binding to corresponding receptors, in accordance with aspects of the invention.

### DETAILED DESCRIPTION

The invention provides systems, including methods, apparatus, compositions, and kits, for connecting cells and/or subcellular structures to substrates, such those defined by coded particles or microplates, among others. The systems may connect members of association pairs to cells/subcellular structures and to substrates, to achieve more stable and/or selective connection of the cells or subcellular structures to the substrates. Such a connection may facilitate multiplexed assays with the cells or subcellular structures. Each association pair may include chemically reactive members (or partners) that react to form one or more covalent bonds. Alternatively, each association pair may include specific binding members that bind one another to form a noncovalent linkage between the members (and thus between cells and the substrate). In some embodiments, a member of the association pair may be connected to external regions of the cells, that is, external to the cell-surface membrane, for example, by linkage to an external polypeptide (or polypeptide domain) or glycan, among others.

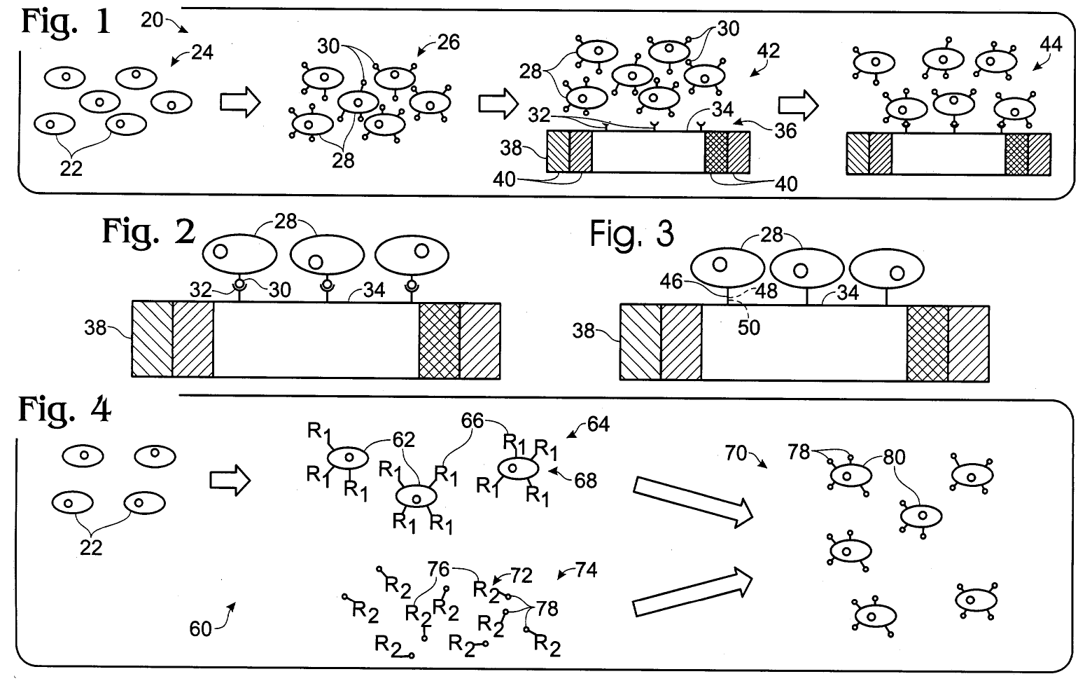

FIG. 1 shows a method 20 of connecting cells to a substrate using an association pair. The method may provide unmodified cells 22, as shown at 24. The unmodified cells may be modified, as shown at 26, to produce modified cells 28 that include an association member 30 of an association pair. Member 30 may be a chemically reactive member or a specific binding member. The association member may be connected covalently or noncovalently to a subcellular structure(s) and/or constituent(s) of the modified cells, such as cell-surface membranes and/or intracellular organelles, among others.

The terms connect, link, and associate, as used herein, have substantially the same meaning, that is, covalent or noncovalent coupling of two or more elements (members, moieties, molecules, a cell(s) and a substrate(s), etc.). When the elements are adjacent one another when connected, such elements are joined (that is, covalently linked or noncovalently coupled). The terms covalently linked and conjugated, as used herein, are intended to have the same meaning, that is, joined by one or more covalent bonds.

Modifying the unmodified cells may be conducted through metabolic and/or nonmetabolic operations. For example, modification may result from feeding unmodified cells 22 with a compound that is internalized and that includes association member 30, a precursor thereof, or a conjugation site therefor. A conjugation site, as used herein, is a region of a cell constituent(s) at which a covalent linkage may be formed. Metabolism may covalently link the association member to a cell constituent, for example, a constituent at least partially disposed in an external region of the cell. Alternatively, modification may be conducted, for example, by introducing a nucleic acid that expresses the association member, a precursor thereof, or a conjugation site therefor. In some embodiments, at least a portion of the modification may be conducted nonmetabolically by contacting the unmodified cells (or partially modified derivatives thereof) with an association member that is linked covalently or that binds to an external region of the cells. Further aspects of cells, association pairs, and connecting members of association pairs to unmodified or partially modified cells are described below, in Sections I, III, and IV, respectively.

In some embodiments, modification of the cells may not be necessary. For example, unmodified cells may present an association member for connection of the cells to a substrate. Such an association member may be a portion or moiety of an external or internal polypeptide, a glycan, a lipid, or the like. Accordingly, in some cases, the association member may be expressed or formed naturally by the cell.

A partner 32 (or second member) of the association pair may be connected to a substrate 34, as shown at 36. The partner may be connected covalently or noncovalently, and connection may be direct or indirect. The substrate may be defined by a coded particle 38 (or a plurality of such particles) having a detectable code 40, as presented here, and/or may be any other assay site, such as microplate well, a test tube, a tissue culture plate, etc. Further aspects of substrates and connecting association members to substrates are described below, in Sections II and IV, respectively.

In some embodiments, the substrate may be defined by at least two classes of coded particles, with each class having a different code. Each class may be connected separately to partner 32, for example, in separate compartments. Alternatively, coded particles of the two or more classes may be mixed before connecting partner 32. Mixing may randomly position the coded particles relative to one another in a nonpositional array. Before or after connecting the partner, one or more coded particles of each class may be placed in a plurality of compartments, such as wells of a microplate. Placing the coded particles may be conducted by distributing portions of a mixture of coded particles or by separately placing the coded particles of each class into the same compartments.

Coded particle(s) 38 and its connected association partner 32 may be contacted with modified cells 28 and their association member 30, shown at 42, to join the association member to its partner by chemical reaction (forming a covalent linkage) and/or binding. As a result, modified cells 28 (or a subset thereof) may become connected to the substrate of coded particle 38, shown at 44, enabling subsequent assays with the connected cells.

Joining may be performed via any suitable mechanism. For example, when performed with a set of two or more classes of coded particles, as described above, joining may be conducted before or after the coded particles are placed in a plurality of compartments. Alternatively, or in addition, joining may be performed in a separate compartment for each different class of coded particles or in a shared compartment with a mixture of the coded particles. Furthermore, joining may connect different sets of cells (such as different types of cells) to different classes of coded particles. Such joining of different sets may be performed in different compartments and/or in the same compartment, for example, by using a different association pair on each set of cells and corresponding class of coded particles.

In some embodiments, modified cells 28 may be connected selectively to the substrate from a larger set of modified and unmodified (or less modified) cells. This selective connection may reduce interference from the unmodified or less modified cells during subsequent assays. Furthermore, in some embodiments, the cells may be fragmented, either before or after connection to the substrate, enabling selective connection of modified subcellular structures to the substrate. Accordingly, interference from unmodified or less modified subcellular structures and/or cellular constituents may be reduced during subsequent assays. Further aspects of contacting substrates with modified cells, and assays that may be performed with cells connected to substrates are described below, in Sections V and VI, respectively.

FIGS. 2 and 3 illustrate different types of connections that may be achieved with association member 30 and partner 32. In these and other figures presented herein, a single site of connection is illustrated between each cell and the substrate to simplify the presentation. However, any suitable number of connection sites may be formed between each cell and a substrate. FIG. 2 shows a noncovalent linkage between member 30 and partner 32, for example, when member 30 and partner 32 form a specific binding pair. FIG. 3 shows a covalent linkage 46 between modified cells 28 and coded particle 38, for example, resulting from conjugation of a chemically reactive association member 48 to its partner 50. Further aspects of connecting cells to substrates are described below, particularly in Section IV and in the examples of Section VII.

The invention provides the potential for increased cell-substrate affinity and enrichment of cells or subcellular structures of interest. For example, the affinity of adherent cells for the substrate may be increased. Alternatively, or in addition, nonadherent cells may be connected to substrates, allowing such cells to be treated and manipulated using adherent-cell methodology. Furthermore, the invention may promote a more effective use of cells, reducing the loss of cells or subcellular structures of interest, while reducing the contribution of other cells or subcellular structures that are not of interest. As a result, the invention may improve signal-to-noise ratios by increasing the number, fraction, and/or density of cells or subcellular structures of interest, thus allowing more compact, efficient, and informative high-throughput screening assays with cells.

Further aspects of the invention are described in the following sections, including (I) cells and subcellular structures; (II) substrates; (III) association pairs, including (A) chemically reactive pairs and (B) specific binding pairs; (IV) connection of association members to cells and substrates; (V) contacting substrates with cells to connect the cells to the substrates; (VI) assays; and (VII) examples.

I. Cells and Subcellular Structures

The invention provides methods for connecting cells or subcellular structures to substrates. Cells generally comprise any self-replicating (and/or self-replicated), membrane-bounded entities that include, or may be modified to include, an association member. Exemplary cells may include whole and/or intact cells, such as eukaryotic or prokaryotic cells, or cell fragments. In addition, exemplary cells may include primary cells, established cell lines, cultured cells, engineered cells, cells directly isolated from nature, etc. Cells may be substantially homogeneous populations or heterogeneous populations, and may be alive or dead. In some embodiments, other biological entities may be used instead of, or in addition to, cells. Such other biological entities may be cell-like, and may include viruses (such as animal virus or phanges), prions, viroids, etc. and/or vesicles.

Cells may be adherent or nonadherent. Adherent cells are any cells that are normally grown and/or manipulated when attached to a substrate. Examples of adherent cells may include fibroblasts, myoblasts, epithelial cells, etc. Nonadherent cells are cells that are not normally grown and/or manipulated when attached to a substrate, for example, cells that grow in suspension. Examples of nonadherent cells may include blood cells, single-celled microorganisms, or other types of suspended cells.

Cells may be modified to include a member of an association pair in an internal region, at the surface, and/or in an external region of a cell. As used herein, internal and external regions of the cell are defined by the disposition of cellular constituents (or domains thereof) in relation to the plasma/surface membrane of such cell. Accordingly, association members that are disposed in an internal region of the cell may be at least substantially bounded by the surface membrane. By contrast, association members that are disposed in an external region of the cell are disposed on a side of the surface membrane that opposes the internal region.

Association members may be connected to subcellular structures and/or cellular constituents. Subcellular structures may include any portion or fragment a cell, generally in the form of an assembly of different cellular constituents. The assembly may be naturally occurring and/or the result of engineering or other experimental manipulation. Examples of subcellular structures may include membranes (such as cell-surface membranes, nuclear membranes, endosomal membranes, lysosomal membranes, etc.) and/or organelles (such as mitochondria, Golgi apparatus, lysosomes, nuclei, secretory granules, nuclear matrices, chromosomes, chloroplasts, nuclear bodies, organelle fragments, or the like), among others. Examples of cellular constituents may include, but are not limited to, polypeptides, glycans (polymers of sugar moieties), lipids, and nucleic acids. In some embodiments, the association member may be conjugated to a polypeptide and/or a glycan in the external region of each cell.

Subcellular structures may be associated with substrates as part of whole cells or as fragments from fragmented cells. Cell fragments may be obtained using any process that breaks cells apart and/or fractionates cells into cellular fractions. Accordingly, cell fragments may be produced from cells by treatment with detergents, mortars and pestles, tissue grinders, sonicators, heat, radiation, pressure, non-physiological concentrations of ions, organic solvents, and/or centrifugation, among others.

Further examples of suitable cells are described in the patent applications identified above under Cross-References, which are incorporated herein by reference, particularly U.S. patent application Ser. No. 10/120,900, filed Apr. 10, 2002.

II. Substrates

Cells and/or subcellular structures may be connected to substrates. Substrates generally comprise any surface or region of a carrier or support that can be connected to a member of an association pair. Substrates may be manufactured, that is, man-made. Accordingly, substrates may be provided or defined by any solid or semi-solid support or carrier, including coded particles, microplate wells (also termed microtiter wells), partitioned or nonpartitioned substantially planar structures (such as microscope slides or cover slips), petri dishes, tissue culture dishes and flasks, etc. Substrates and/or their supports/carriers may be composed of, and/or may include, any suitable material, including glass, ceramic, plastic, metal, silicon, carbon, protein, glycan, extracellular matrix components, etc.

Coded particles may be connected to cells. Such coded particles generally comprise populations of particles, distinguishable at least in part by a detectable code. Each particle includes a substrate and a connected code. Accordingly, the code may identify the substrate, the cells connected to the substrate, modulators exposed to the cells, experimental manipulations, etc. The particles may have any suitable composition, size, and shape consistent with an ability to perform their intended function.

Particles may have a composition that includes glass, plastic (such as a polyacrylate), ceramic, sol-gel material, metal, protein, nucleic acid, lipid, and/or glycan, among others. The material may be a solid, a gel or other porous material, and/or a combination thereof. In some embodiments, the particles may include a core portion, such as glass or plastic, among others, and a material connected to the core portion. Accordingly, the core portion may include the code and may be inanimate.

The particles generally may have any suitable size. Preferred properties are determined by the application. For example, preferred sizes may be determined in part by what the particles are connected to and identify, with particles preferably being at least a few times larger than the molecules, organelles, viruses, cells, and/or so on that the particles may be connected to and support. Preferred sizes also may be determined in part by the detection method, with particles preferably being (at least for optical detection) larger than the wavelength of light but smaller than the field of view. Preferred sizes provide particles termed microparticles. Microparticles may range between about ten microns and about four millimeters in length. Alternatively, or in addition, microparticles may have a length related to the cells connected to the particles, with the (average) length of the microparticles being greater than the (average) diameter of the cells, or between about one to fifty cell diameters, among others.

Numerous applications of the invention may be carried out in microplates or similar sample holders having a relatively high density of relatively low volume wells. In these applications, the microparticles preferably should be small enough so that at least two or more microparticles may be viewed in the well simultaneously. Therefore, the maximum size dimension for microparticles sometimes may be dictated by the well dimension in a specific microplate configuration or density. Conversely, the minimum area of microparticles preferably should be large enough to support at least one cell. Thus, microparticles for multiplexed cellular experiments may have an area of at least about 100 square microns.

The particles generally may have any suitable geometry. Preferred particle geometries may include at least substantially planar, for example, in the form of a wafer or sheet, and at least substantially cylindrical. The wafer or sheet may be square, rectangular, polygonal, circular, elliptical, and/or curvilinear, among others, when viewed from the top, side, or end, and may have at least one pair of opposing surfaces that are generally parallel. In some embodiments, at least one surface provides an experimental platform for performing an assay. In some embodiments, the particles may include one or more recesses, ridges, and/or grooves at their surfaces or may have smooth surfaces.

The code generally comprises any mechanism capable of distinguishing different particles. The code may relate to overall features of the particles. These features may include particle size, shape, and/or composition. Alternatively, or in addition, the code may relate to subfeatures of the particle. These subfeatures may be positional and/or nonpositional, meaning that the code may be based on the presence, identities, amounts, and/or properties of materials at different positions in the particle and/or at potentially the same position in the particle, respectively. These positions may be random and/or predefined.

Exemplary positional and nonpositional codes may be optically detectable. Such codes may be formed by using materials that differ in how they generate and/or interact with light (i.e., electromagnetic radiation, particularly visible light, ultraviolet light, and infrared light), such as their absorption, fluorescence, diffraction, reflection, color (hue, saturation, and/or value), intrinsic polarization, chemiluminescence, bioluminescence, and/or any other optically distinct property or characteristic. Positional codes may be formed by positioning different amounts and/or types of materials at different positions in or on a particle, for example, at spots, lines, concentric circles, and/or the like. These positional codes may be read by determining the identities, amounts, and/or other properties of the code materials at each code position, for example, by measuring intensity as a function of position. Nonpositional codes may be formed, for example, by using at least two different materials, potentially at the same position, where the materials differ in how they interact with light. These nonpositional codes may be read by determining the presence and/or other properties of signals from the different materials, for example, by measuring intensity as a function of wavelength for an optical code. In each case, the amounts, positions, and/or values may be relative or absolute. Moreover, different types of codes may be combined to form yet other types of codes. In some embodiments, the codes may be read directly by interrogation with light (electromagnetic radiation), without a need to react or process the particles to reveal the codes.

Codes may define classes of particles. Each particle class is defined by a different code (or set of codes). Accordingly, a set of coded particles may include at least two different classes with different codes. As a result, particles in different classes may be connected to different cells, may have different compositions, and/or may be manipulated differently, among others. The different cells, compositions, and/or manipulations may be identified by the different code. Coded particles (also termed coded carriers)—including particles, codes, and nonpositional arrays of coded particles—are described in more detail in the patents and patent applications listed above under Cross-References and incorporated herein by reference, particularly the following U.S. patent application Ser. No. 09/694,011, filed Oct. 19, 2000; Ser. No. 10/120,900, filed Apr. 10, 2002; and Ser. No. 10/273,605, filed Oct. 18, 2002.

Wells or sub-wells of microplates may be connected to cells. As used herein, microplates may include any sample holder that defines a fixed array of distinct compartments for holding fluid. The microplates may have any suitable number of compartments or wells, such as 24, 96, 384, or 1536, among others. In some embodiments, the microplates may have wells that are subdivided into sub-wells. The sub-wells of each well may be in adjustable fluid communication, so that the sub-wells may be addressed separately or together, as desired, based on the volume of fluid added to the corresponding well. Microplates, wells, and sub-wells are described further below, in Example 4, and in the patent applications listed above under Cross-References and incorporated herein by reference, particularly U.S. patent application Ser. No. 10/282,940, filed Oct. 28, 2002.

III. Association Pairs

Cells may be connected to substrates by association pairs. As used herein, an association pair generally includes a pair of moieties and/or molecules that can be linked directly to each other. Such linkage may be a covalent linkage, that is, one or more covalent bonds may be formed between the members of the association pair by chemical reaction. Accordingly, the members may form a chemically reactive pair, in which both members of such a pair may be chemically modified by formation of the covalent linkage. Alternatively, or in addition, such linkage may be a noncovalent linkage formed by physical interaction or binding between the members of the association pair. In this case, the members may form a specific binding pair.

An individual connection between a cell and a substrate may be formed by any suitable combination of covalent and noncovalent linkages. For example, the connection may be the result of forming one or more covalent linkages by chemical reaction of a corresponding one or more chemically reactive pairs, the result of one or more physical interactions between a corresponding one or more specific binding pairs, and/or a combination of such chemical reactions and physical interactions. The covalent linkages and binding interactions may be formed sequentially, in any suitable order, or at least substantially at the same time. In addition, the cell may be connected to the substrate by any suitable number of such individual connections.

A. Chemically Reactive Pairs

Chemically reactive pairs may include any pair of moieties and/or molecules that can be covalently linked by formation of one or more covalent bonds between the moieties and/or molecules. Formation of the covalent bonds may be specific, that is, representing the majority of covalent bond formation in a reaction, or may be nonspecific, that is, representing the minority of such bond formation.

Table 1 shows various exemplary reactive moieties or members of chemically reactive pairs, and covalent linkages produced by chemical reaction of such moieties. The reactive moieties may be classified as electrophilic and nucleophilic to indicate propensity to accept and donate electrons, respectively. However, in some embodiments, the chemically reactive pairs may react through joining of free radicals to form one or more bonds, and thus may be equally nucleophilic and electrophilic. Reactive moieties may be included in any suitable molecules with any other suitable functional groups. Reactions between members of chemically reactive pairs may be conducted in any suitable reaction environment, that is, aqueous or nonaqueous fluids, at any suitable pH, ionic strength, and temperature, and for any suitable time. In addition, such reactions may be catalyzed by the addition of any suitable chemical catalyst or physical catalyst (such as light).

TABLE 1
Chemically Reactive Association Pairs

| Electrophilic Moiety | Nucleophilic Moiety | Resultant Covalent Linkage |
|---|---|---|
| activated esters | amines/anilines | carboxamides |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazides | hydrazones |
| aldehydes or ketones | hydroxylamines | oximes |
| aldehydes or ketones | thiosemicarbazides | thiosemicarbazones |
| alkyl halides | amines/anilines | alkyl amines |
| alkyl halides | carboxylic acids | esters |
| alkyl halides | thiols | thioethers |
| afryl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiol | thiophenols |
| aryl halides | amines | aryl amines |
| azindines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | ammotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

B. Specific Binding Pairs

Specific binding pairs (SBPs) may include any pair of moieties and/or molecules (such as first and second specific binding members) that bind selectively to each other, typically with high affinity, and generally to the exclusion of binding to other moieties.

The binding between members of a specific binding pair may be driven by any suitable physical interaction(s), including but not limited to electrostatic, charge-charge, or ionic interactions, van der Waals interactions, hydrogen-bonding interactions, hydrophobic-hydrophilic interactions, dipole-dipole interactions, and/or the like. These interactions generally do not require covalent interactions; however, these interactions may, in some cases, be supplemented by such interactions, for example, using cross-linking reagents.

The members of a specific binding pair (e.g., a specific binding member and its partner) may be connected to, or disposed on, cells (and/or subcellular structures) and substrates, respectively.

The specific binding between members of a specific binding pair can be characterized by a binding coefficient. Generally, such specific binding coefficients range from about 10⁻⁴ M to about 10⁻¹² M or 10⁻¹⁴ M and lower, and preferred specific binding coefficients range from about 10⁻⁵ M, 10⁻⁷ M, or 10⁻⁹ M and lower.

Table 2 shows various exemplary specific binding pairs.

TABLE 2
Representative Specific Binding Pairs

| First Member | Second Member |
|---|---|
| biotin | avidin or streptavidin |
| antigen | antibody |
| carbohydrate/glycan | lectin or carbohydrate receptor |
| DNA | antisense DNA; protein |
| enzyme substrate | enzyme; protein. |
| histidine | NTA (nitrilotriacetic acid) |
| IgG | protein A or protein G |
| RNA | antisense or other RNA; protein |
| hormone | hormone receptor |
| ion | chelator |

Here, the designators “first” and “second” are arbitrary relative to the use of those terms elsewhere in this disclosure, such that these terms may be interchanged as desired or warranted. The exemplary specific binding pairs may include natural high-affinity pairs. For example, biotin and avidin or streptavidin may be utilized to connect cells to substrates. Alternatively, one member of a specific binding pair may be a single-chain antigen-binding domain, and its partner may be a corresponding antigen, for example, as described in U.S. Pat. No. 6,017,754 to Chestnut et al., which is incorporated herein by reference. Furthermore, the specific binding member and its partner may be an encoded receptor or ligand, such as EGF receptor/EGF or IL-1 receptor/IL-1, etc. Further examples of the use of biotin and avidin to connect cells to substrates are described below, in Section VII.

IV. Connection of Association Members to Cells and Substrates

First and second members of an association pair may be connected to cells and substrates, to enable subsequent connection of the cells to the substrates by joining the members of the pair. Connection of the first and second members to the cells and substrates may be covalent and/or noncovalent. In addition, the connection may link either member of the association pair to the cells, to modify the cells, with the member's partner connected to the substrate(s). For example, an electrophilic association member may be connected to cells and a nucleophilic partner of the electrophilic member connected to a substrate, or vice versa.

Modification of cells by connection of a member of an association pair to the cells may be nonmetabolic and/or metabolic. Alternatively, as described above, cells may include a suitable association member without modification.

Nonmetabolic connection, as used herein, involves covalent or noncovalent linkage of a member of an association pair to cells through contact of the member (or a precursor thereof) with the external region of the cells (or fragments thereof). Accordingly, nonmetabolic connection may be conducted on living or dead cells without a need for introduction of the association member (or precursor) into the internal region of the cells. Exemplary nonmetabolic connection may include direct binding of a specific binding member to an external region of the cells (or cell fragments) and/or covalent linkage of a chemically reactive association member to a partner moiety disposed in the external region.

Metabolic connection, as used herein, involves covalent or noncovalent joining of an association member to a constituent of cells, at least partially as a result of one or more enzymatic processes of the cells. In some embodiments, the connection may involve conjugation (covalent joining) of the association member to a conjugation site of the cell constituent. The enzymatic processes may involve transport of the association member into the cell, production of the association member (such as through transcription or translation, among others), conjugation of the association member or precursor to the conjugation site of the cell constituent (such as by ligation, polymerization, or other chemical reaction), structural modification of the precursor to form the association member, and/or the like. In some embodiments, cells may be engineered to include a constituent having a conjugation site for the association member. Alternatively, or in addition, the cells may be engineered to include a conjugating activity (such as a ligase) to conjugate an association member to a cell constituent. Examples 1 and 2 below describe sequential metabolic and nonmetabolic connection of association members to cells.

The association member, precursor, and/or conjugation site may be disposed inside cells, to enable metabolic connection, by any suitable mechanism. For example, the member, precursor, or conjugation site may be included a compound that is introduced into cells from fluid surrounding the cells (such as the growth medium). Such introduction may be mediated by transport processes, transfection, shock loading, poration, etc. Alternatively, the association member, precursor, and/or conjugation site may be synthesized by the cells naturally or by engineering (for example, as a result of transfection). In some embodiments, a combination of these routes may be used. For example, an association member may be introduced into the cells from the external fluid and then conjugated to (and/or incorporated into) a cellular constituent by a natural and/or engineered enzymatic process(es).

In some embodiments, cell-restricted expression of the association member, the conjugation site, and/or a conjugating activity may determine which cells and/or subcellular structures are connected with higher affinity and/or selectivity to the substrate. For example, uptake, synthesis, and/or conjugation of the association member (and/or conjugation site) may be selective for specific cells, based on cell type, relative rates of growth, transfection of particular cells, etc. Example 5 below describes further aspects of selectively connecting transfected cells to substrates.

In some embodiments, a compound(s) that is a derivative of a cell component(s) may be introduced into cells. For example, the derivative may be related to a natural cell nutrient and/or metabolite by conjugation of such nutrient and/or metabolite to an association member (or a precursor thereof) and/or may form a conjugation site for the association member. Exemplary derivatives may be components of polymers, such as amino acids, sugars (saccharides), nucleotides, etc. Accordingly, the polymers may be polypeptides, glycans, polynucleotides, and/or the like.

The invention may provide a kit for connecting particles to cells. The kit may include a reagent configured to covalently link an association member to an external region of the cells. The reagent may include any suitable form of the compound described above, and fluid, salts, additives, stabilizers, cell nutrients, etc. The kit also may include a set of coded particles having the partner of the association member connected to the coded particles. The kit also may include instructions, sample containers with separate compartments for performing simultaneous and/or sequential assays, etc.

In some embodiments, the association member may be included in a saccharide derivative that is connected metabolically to cells. The saccharide derivative may include a chemically reactive member of an association pair, such as a ketone, a hydrazide, a hydroxylamine, a thiosemicarbazide, or any other suitable member of Table 1. The saccharide derivative may be taken up by the cells from the surrounding fluid and conjugated to a glycan that is later disposed in an external region of the cells. Further aspects of the use of saccharide derivatives are described below in Example 2.

In some embodiments, expression of the association member and/or conjugation site may be restricted by nucleotide control sequences that are cell-selective or conditionally active, for example, responsive to specific signaling pathways, cell milieus, or cell identities, among others. As a result, in some embodiments, cells that activate expression of a transfected nucleic acid may be connected selectively to the substrate.

Subcellular localization of an association member (or precursor and/or a conjugation site) also may determine the cells and/or subcellular structures that are more stably and/or selectively connected to the substrate. The association member may be localized to a subcellular structure(s) through localizing signals or domains. Localizing domains generally comprise molecular addresses and/or interaction motifs that position the association member in, on, or about the cell. Molecular addresses include signal sequences, membrane-spanning domains, nuclear localization signals, and so on. Interaction motifs include any motif that binds with high affinity to a positioned cell component. Examples of positioned cell constituents include nuclear histones, polymerized actin, microtubules, nuclear pore components, membrane-associated or -spanning components, and so on.

The association member may be expressed as part of, or conjugated to, a protein, such as a fusion protein. The protein may include targeting or localizing sequences that direct the protein, and thus the association member, to a specific subcellular structure. For example, the protein may be targeted to the cell surface, such as with a secretion signal and a membrane-spanning or -associated domain, to facilitate connection of whole cells to substrates. Such a protein also may allow cell-surface membranes to be connected selectively to the substrate after cell disruption, generally enriching for cell-surface membranes over other subcellular structures and to enrich for membranes from modified over unmodified cells. Alternatively, or in addition, the protein may be targeted to other subcellular structures, such as nuclei, chromosomes, internal membranes, or so on.

Connection of an association member to a substrate may be performed during and/or after manufacture of the corresponding support/carrier that defines the substrate. Connection during manufacture may be performed, for example, by forming the support/carrier of a material that includes an association member. For example, the support/carrier that defines the substrate may be formed of a polymer that includes ketone or hydrazide/hydroxylamine moieties, which may form covalent linkages with hydrazide/hydroxylamine and ketone moieties, respectively, connected to cells or connected to another association member. Connection after manufacture may be performed, for example, through nonspecific interactions, interaction of specific binding pairs, and/or covalent attachment, among others. For example, the substrate may be chemically modified to include ketone, hydrazide, hydroxylamine, or thiosemicarbazide moieties, among others. Alternatively, such moieties may be carried by a molecule, such as polylysine, that may be connected to the support/carrier by nonspecific or specific interactions.

V. Contacting Substrates with Cells to Connect the Cells to the Substrates

Cells and substrates connected to members of an association pair may contact one another to promote covalent or noncovalent linkage of the members. The linkage may join the members, thereby connecting the cells to the substrates. Contacting may be performed in solution or suspension or on a surface, in or on any suitable container or other support, and under any suitable conditions to form the connection between the cells and the substrates.

Contacting may connect modified cells selectively relative to unmodified cells, or modified subcellular structures relative to unmodified subcellular structures. Contacting may be carried out by combining the cells or subcellular structures with a substrate, and then incubating for a suitable time period. Separating connected and unconnected cells or subcellular structures from each other may be performed subsequently.

The contacting operation may include the use of reagents and/or conditions that facilitate chemical reaction or binding of association members, as appropriate. In some embodiments, this operation may be carried out at a pH, ionic strength, and/or concentration that disfavors normal cell-substrate interaction, but that permits chemical reaction or physical interaction of association pairs connected to the cells/structure and substrate. For example, the substrate may lack components that normally assist cell-subtrate interactions, such as poly-L-lysine or extracellular matrix material, and/or may be of a material, such as untreated plastic, that generally does not promote cell binding. Alternatively, or in addition, the substrate may include such components, but the cells and substrate may be combined/incubated in the presence of excess, unbound cell adhesion material, such as poly-L-lysine or extracellular matrix material.

The separating operation also may favor retention of cells that are connected to the substrate through an association pair. Thus, the separating operation may include multiple washes and may be performed under conditions that disrupt or weaken normal cell-substrate interactions.

VI. Assays

Assays may be performed on cells or subcellular structures that are connected to the substrate(s). As used herein, an assay includes any analysis of (or with) the cells or structures. The assay may measure the efficiency or amount of connection between the cells/structures and substrate(s). For example, the assay may measure the number of cells bound to one or more substrates. Alternatively, the assay may measure a characteristic of the cells/structures that is different from connection to the substrate.

The characteristic generally comprises any measurable aspect of the cells or subcellular structures. Examples of characteristics include cell phenotype, cell growth, cell identity, cell morphology, apoptosis, cell spreading, motility, intracellular trafficking, expression of an endogenous or exogenous gene, binding of a ligand, effect of a test compound or candidate modulator, level or location of a protein, and/or so on.

The characteristic may be related to cell modification, and thus may be a measure of the effect of a specific nucleic acid that was introduced into the cells. For example, when cells are modified, they may be co-transfected with nucleic acid encoding a protein of interest or effector, such as a cell-surface receptor, among others, and/or with a reporter gene.

Assays, particularly multiplexed assays, may be performed with coded particles. As used herein, multiplexed assays involve two or more assays conducted together in a shared compartment. In such assays, individual particles and their connected cells may be identified by reading the code of the individual particles. Cell characteristics may be measured and codes may be read at any time during a multiplexed assay with coded particles. Measurement of cell characteristics and reading codes may be performed in any order and on any number of cells and particles. Moreover, these steps generally may be performed using any suitable examination site, such as a slide, a microplate, or a capillary tube, and any suitable detection device, such as a microscope, a CCD array, an optical sensor, a film scanner, or a plate reader.

The cell characteristic may be measured from at least one, and up to all, of the cells connected to a particle. Accordingly, the characteristic may relate to individual cells, subcellular regions of the subset, or extracellular regions adjacent individual cells. The characteristic may be measured from only one cell or region connected to a particle, from less than all cells or regions connected to a particle, and/or from all cells or regions connected to a particle.

The characteristic may be any molecular, cellular, and/or extracellular aspect measured from cells connected to particles. The molecular characteristic may relate to a cellular constituent, such as the number, concentration, distribution, presence/absence, partnership, structure, modification, or activity (such as enzyme activity or binding activity) of the constituent. Exemplary constituents may include a nucleic acid, protein, ion (for example, to measure calcium flux), lipid, carbohydrate, metabolite, etc. Exemplary proteins may include reporter proteins that are encoded by the transfection material (such as beta-galactosidase, GFP, luciferase, chloramphenicol acetyl transferase, etc.). The constituents may be endogenous, transfected, or encoded, among others. Cellular aspects may include any measurable cellular or subcellular phenotype, such as cell proliferation, reporter gene activity, cell cycle distribution, DNA synthesis, nuclear import, signal transduction, differentiation, transcription, morphology, apoptosis, import, export, subcellular transport, electrical activity, and/or the like.

Cell characteristics may be measured adjacent any suitable surface or surfaces of the particle to which the subset is apposed (connected). The subset may be apposed to one surface of the particle, opposing surfaces of the particle, and/or to any selected subset or set of surfaces of the particle, among others. Accordingly, cell characteristics may be measured for a subset of cells apposed to one particle surface, opposing surfaces, and/or near any region of the particle.

The code may be read before, during, and/or after measuring the cell characteristic. Reading the code may include discerning or determining a positional and/or nonpositional code of a particle by any suitable approach, such as optical and/or nonoptical techniques. Exemplary optical techniques include sensing light (particularly visible light, UV light, and infrared light) positionally or nonpositionally from a particle. Exemplary nonoptical techniques may include electrical analysis of a particle to read a nonoptical code, such as measurement of the particle's capacitance, impedance, conductance, etc., in a positional or nonpositional fashion within the particle. Whenever the code is read, it should be linked or linkable to the measured cell characteristic or interaction. This linkage may identify the transfection material associated with the particle, and/or may identify other aspects related to the particle, including the type of cells, the modulator exposed to the cells, other experimental parameters (such as reaction times or conditions), order of manipulations, and/or so on.

Additional exemplary assays, cell characteristics, and methods for reading codes and measuring cell characteristics, are described in more detail in the patents and patent applications identified above under Cross-References, which are incorporated herein by this reference, particularly the following U.S. patent application Ser. No. 09/694,077, filed Oct. 19, 2000; Ser. No. 10/120,900, filed Apr. 10, 2002; and Ser. No. 10/282,904, filed Oct. 28, 2002.

### VII EXAMPLES

The following examples describe selected aspects and embodiments of the invention. These examples include methods of connecting cells or subcellular structures to substrates by utilizing chemically reactive and/or physically binding association pairs, and assays with the connected cells or subcellular structures. These examples are included for illustration and are not intended to limit or define the entire scope of the invention.

### Example 1

Sequential Modification of Cells

This example describes a general method 60 for sequential modification of cells with association members; see FIG. 4.

In method 60, unmodified cells 22 may be modified in a first modification, to produce reactive cells 62, shown at 64. Reactive cells 62 may include a first association member 66 (“R₁”) that is chemically reactive. R₁ may be an electrophile or a nucleophile, among others. In addition, R₁ may be covalently linked to a cell constituent (or domain thereof) disposed in an external region 68 of the cells. Such covalent linkage may be performed metabolically or nonmetabolically, as described above in Section IV. In alternative embodiments, R₁ may be linked noncovalently to external region 68 of the cells.

Reactive cells 62 may be further modified by a nonmetabolic process, shown at 70. Reactive cells 62 may be contacted with a reactive molecule (or complex) 72 that is bifunctional, shown at 74. Reactive molecule 72 may include a reactive partner 76 (“R₂”) of R₁ and a specific binding member 78, which are linked covalently or noncovalently to one another. Accordingly, reaction of R₁ with R₂ may connect specific binding member 78 to modified cells 80. Here, the connection is a covalent linkage.

### Example 2

Connection of Cells to Coded Particles through Surface Glycans

This example describes a method 90 of connecting cells to coded particles by metabolically conjugating a chemically reactive association member to glycans disposed in an external region of cells; see FIG. 5.

Cells 22 are metabolically labeled, shown at 92. The cells may be contacted with a saccharide derivative 94 that includes a chemically reactive association member 96. The saccharide derivative may be a monosaccharide, such as mannose, glucose, fucose, etc., or may be a di- or polysaccharide, among others. Reactive member 96 of the saccharide derivative may be, for example, a ketone group (“K”). Accordingly, the saccharide derivative may be N-levulinoyl mannosamine (ManLev). Alternatively, the ketone group may be replaced by any suitable reactive group or specific binding member (as exemplified in Tables 1 and 2 above), for example, a hydrazide, a thiosemicarbazide, or hydroxylamine, among others. Internalization and metabolism of the saccharide derivative may covalently link the saccharide to other saccharides, to produce a reactive glycoconjugate or glycan. The glycoconjugate or glycan may be targeted for expression on the cell surface, such that it is connected to cells 22 in an external region of such cells, to produce reactive cells 98. The glycoconjugate or glycan may be included in a glycoprotein, a proteoglycan, and/or a glycolipid, among others, of the cells.

Reactive cells 98 may be further modified, shown at 100. For example, the reactive cells may be contacted with a bifunctional reactive molecule or complex 102. Molecule or complex 102 may be bifunctional, including a reactive partner 104 (“*”) of reactive member 96 linked (covalently or noncovalently) to a first specific binding member 106, such as biotin (“B”). Accordingly, the reactive member and partner may react, shown at 108, to link the first specific binding member to modified cells 110, in an external region of the cells. Here, the linkage is covalent. Alternatively, or in addition, the reactive cells (in this or other embodiments) may be treated with agents and/or conditions that enhance the accessibility of association partners. Such agents may include enzymes, such as proteases and/or carbohydrases, among others, that remove cell surface components that otherwise may inhibit association between association partners.

Modified cells 110 may be connected to coded particle(s) 38, as shown at 112. The coded particle may be connected to a second specific binding member 114, such as avidin (“A”), so that the second binding member is accessible to the cells. Modified cells 110 may contact the coded particle to enable the first and second specific binding members to bind one another. In some embodiments, a plurality of different sets of modified cells may be connected separately to different classes of coded particles having different codes. Subsequently, the different classes and their connected cells may be mixed to perform assays on the cells.

Further aspects of connecting association members to glycans, such as other saccharide derivatives that may be suitable and specific reaction conditions, are included in U.S. Pat. No. 6,075,134, and U.S. Pat. No. 6,458,937, each issued to Bertozzi et al. and incorporated herein by reference. Additional aspects are also included in the following refererences, which are incorporated herein by reference: “Engineering Chemical Reactivity on Cell Surfaces Through Oligosaccharide Biosynthesis,” Science, May 16, 1997, Vol. 276, pp. 1125-1128; “Metabolic Delivery of Ketone Groups to Sialic Acid Residues. Application to Cell Surface Glycoform Engineering,” The Journal of Biological Chemistry, Nov. 20, 1998, Vol. 273, pp. 31168-31179; and “Engineering Novel Cell Surface Receptors for Virus-Mediated Gene Transfer,” The Journal of Biological Chemistry, Jul. 30, 1999, Vol. 274, No. 31, pp. 21878-21884.

### Example 3

Covalent Connection of Metabolically Modified Cells to Coded Particles

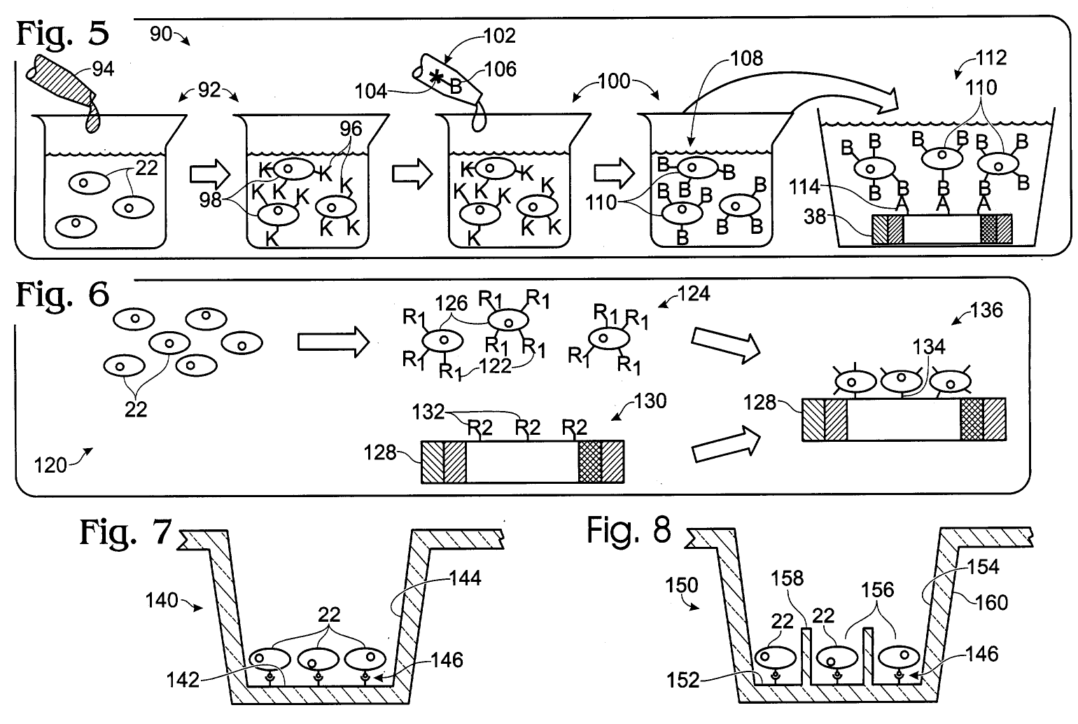

This example describes a method 120 of covalently connecting cells to coded particles; see FIG. 6.

Cells 22 may be modified by metabolically conjugating a chemically reactive association member 122 (“R₁”) to an external region of the cells, shown at 124. Accordingly, reactive member 122 may be conjugated, for example, by metabolically labeling the cells with a reactive saccharide derivative, as described in Example 2, to produce reactive cells 126.

Reactive coded particle 128 may be provided, shown at 130, by connecting a reactive partner 132 (“R₂”) to the particles. The reactive partner may be connected covalently or noncovalently. The reactive particles may be prepared at any suitable time relative to preparation of reactive cells 126.

Reactive particle 128 may be contacted with reactive cells 126, to enable chemical reaction between reactive member and partner 122, 132. Chemical reaction forms covalent linkages 134 between the cells and the particles, shown at 136.

### Example 4

Connecting Cells to Microplate-Based Substrates

This example describes connecting cells to microplate-based substrates, including wells and sub-wells, using association pairs; see FIGS. 7 and 8.

FIG. 7 shows a system 140 for microplate-based connection of cells 22 to a substrate 142 provided by a microplate well 144. Any suitable association pairs 146, as described above, may be used to connect the cells to the substrate. System 140 may be particularly suitable for nonadherent cells, which may be difficult to assay otherwise in a microplate format. System 140 also may enable more efficient retention of cells 22 on substrate 142 during fluid manipulations, may enable fewer cells to be used in an assay, and/or may facilitate selection or enrichment of cells of interest from a mixed cell population.

FIG. 8 shows another system 150 for microplate-based connection of cells 22 to a substrate 152 provided by a well 154 of a microplate. However, in system 150, wells 154 are subdivided into sub-wells 156 by subdividing walls 158. Walls 158 may have a lower height than surrounding well wall 160, so that the volume of fluid added to the wells allows sub-wells to be addressed individually or together. Accordingly, cells may be connected in fluid isolation or fluid communication to substrate 152 of each sub-well 156. Similarly, the connected cells may be contacted with modulators (such as test compounds) in fluid isolation (for example, with different modulators), or together, in fluid communication (for example, with the same modulator). Exemplary microplates having subdivided wells are described in U.S. patent application Ser. No. 10/282,940, filed Oct. 28, 2002, which is incorporated herein by reference.

### Example 5

Connection of Transfected Cells to Coded Particles using Biotin and Avidin

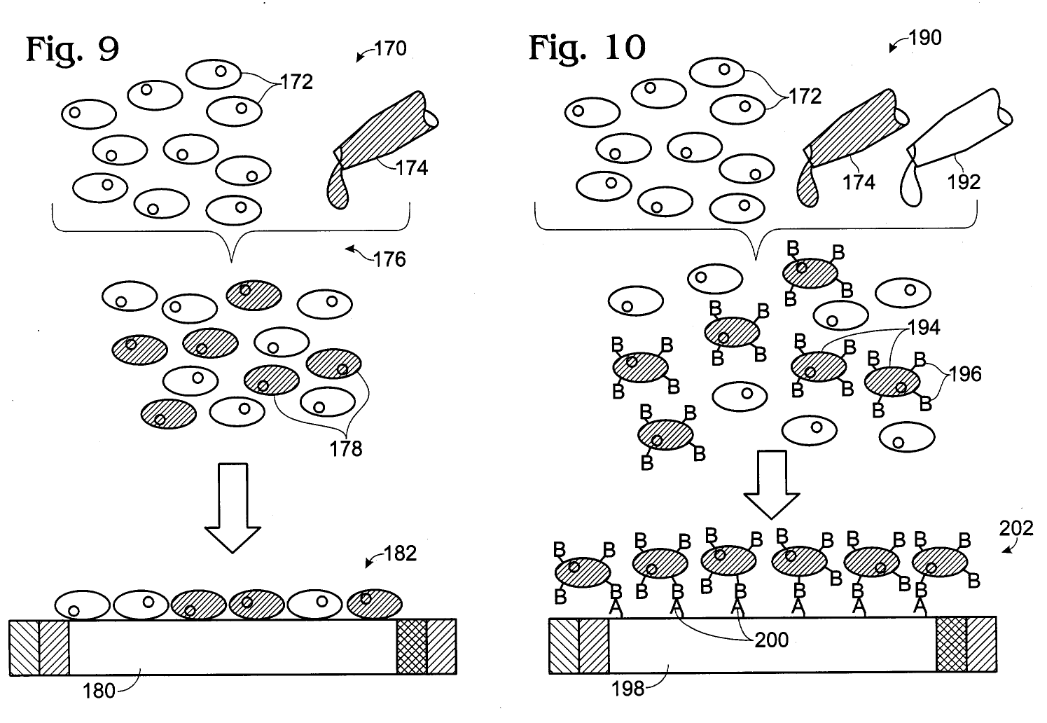

This example compares methods for nonselective and selective association of a transfected cell population with coded particles, using interaction between biotin and avidin (or streptavidin); see FIGS. 9 and 10.

Cells may be connected to substrates using biotin binding to avidin or streptavidin. The biotin-avidin interaction not only is well characterized but also is one of the strongest molecular interactions known in biochemistry: biotin binds to avidin with an estimated binding coefficient of less than 10⁻¹⁴ M. Biotin may be synthesized by cells and/or introduced by uptake from the extracellular milieu during growth and/or incubation of cells. Thus, biotin includes any naturally occurring or synthetic variants of biotin that retain an ability to interact strongly with avidin and/or its derivatives/relatives, as described below.

Biotin may be covalently conjugated to biotin acceptor sites (conjugation sites) in both prokaryotic and eukaryotic cells, using an endogenous biotin ligase activity. Examples of naturally occurring biotin acceptor sites that may be suitable are found in carboxylases, decarboxylases, transcarboxylates, or subdomains thereof, among others, as described in U.S. Pat. No. 5,252,466 to Cronan, which is incorporated herein by reference. In some embodiments, the biotin acceptor site may be derived from the 1.3S subunit of a transcarboxylase from Propionibacterium shermanii, termed PST. The PST biotin acceptor site may include a C-terminal portion of the 1.3S subunit, such as about 75 or about 70 amino acids of the carboxyl terminus. Alternatively, the biotin acceptor site may include a sequence identified by peptide screening techniques, such as biotin acceptor sites described in U.S. Pat. No. 5,723,584 to Schatz. With a natural and/or experimentally derived acceptor site, the site may be altered by substitutions, deletions, and/or insertions that do not substantially disrupt ability to direct biotin conjugation. For example, cells may be transfected with a vector that expresses a fusion between the 1.3S subunit of the transcarboxylase of P. shermanii (PSTCD, which acts as a biotin acceptor) fused with the transmembrane domain of the platelet-derived growth factor receptor (PDGFtm, which acts as a particle to take the biotinylated PSTCD to the cell surface), resulting in the expression of a biotinylated form of the fusion protein (PSTCD-PDGFtm) on the cell surface. Further aspects of biotin fusion proteins are discussed in Parrott, M. B., and Barry, M. A. (2001) Biochem. Biophys. Res. Comm. 281, 993-1000, which is incorporated herein by reference.

With biotin as the specific binding member, a suitable high affinity partner is avidin from vertebrates, streptavidin from Streptomyces, or any derivatives or relatives of avidin or streptavidin. Avidin, streptavidin, and derivatives or relatives thereof, may be produced metabolically and/or synthetically. Derivatives/relatives may include mutants (for example, substitutions, deletions, insertions, truncations, and/or chimeras, among others), avidin/streptavidin-like polypeptides identified from other species, and/or chemically modified forms of avidin or streptavidin.

An association member may be conjugated posttranslationally to a biotin acceptor site using an endogenous or exogenous conjugating activity. For example, biotin may be conjugated to biotin acceptor sites using an endogenous biotin ligase activity. Alternatively, biotin may be conjugated by expressing a biotin ligase, such as BirA from E. coli, from an exogenous expression vector. The exogenous expression vector may be introduced by co-transfection, along with the nucleic acid encoding the conjugation site, and may be a distinct nucleic acid molecule or may be included in the nucleic acid encoding the conjugation site. Furthermore, the encoded conjugating activity may be a fusion protein that is targeted or localized to a subcellular structure or region based on localizing domains or signals fused to the conjugating activity. For example, BirA may be targeted to a cell's secretory pathway by fusing a signal sequence, thus potentially increasing biotinylation for secreted/transmembrane modifying proteins. Regulatory sequences that control expression of the conjugating activity may be active in many cell types or may be cell-restricted or cell-specific in their activity. Accordingly, a selectively expressed conjugating activity may be used to limit conjugation and subsequent substrate connection to a subset of the cells (for example, specific cell types of cells) in a mixed cell population.

In some embodiments, the conjugation site may be included in a polypeptide having a signal sequence for entry into a cell's secretory pathway and a transmembrane domain to interrupt secretion and retain a portion of the polypeptide in the membrane. For example, a signal sequence may be positioned near or at the amino terminus of the polypeptide, followed by a biotin acceptor site and then a transmembrane domain. Posttranslational biotinylation of the acceptor site may dispose biotin in the exterior region of the cells. Signal sequences and transmembrane domains that may be suitable are described in U.S. Pat. No. 6,017,754, to Chestnut et al., which is incorporated herein by reference.

FIG. 9 shows a method 170 of nonselective connection of transfected cells to coded particles. Cells 172 may be transfected with a transfection material, such as a nucleic acid 174, shown at 176. The transfection material may transfect a subset of the cells to produce a transfected or modified group of cells 178 (shown as hatched). Modified cells 178 may include a nucleic acid configured to decrease expression of a gene of interest, may include or express a new protein, may overexpress a pre-existing protein, or may exhibit some other change in composition determined by nucleic acid 174. However, the ability of modified cells 178 to be connected to coded particle 180 may be substantially unchanged. Accordingly, both transfected and untransfected (modified and unmodified) cells may be connected with substanially equal efficiency, shown at 182, so that binding is nonselective.

FIG. 10 shows a method 190 of selective connection of transfected cells to coded particles. Cells 172 may be transfected with first and second nucleic acids 174, 192, in parallel or sequentially (or with nucleic acid 192 alone). First nucleic 174 has been described above in relation to method 170 (FIG. 9). Second nucleic acid 192 may be configured to promote biotin conjugation adjacent the surface of cells 172. More particularly, the second nucleic acid may encode a membrane-associated protein. The protein may include a biotin acceptor site disposed in an external region of the cells after trafficking of the protein to the membrane and proper placement of the protein relative to the membrane. Accordingly, biotin may be conjugated to the biotin acceptor site to produce biotinylated cells 194 having a plurality of biotin moieties 196 (“B”) connected to the cells.

Coded particle 198 connected to avidin or streptavidin moieties 200 (“A”) may be contacted with biotinylated cells 194, shown at 202. The biotinylated cells may be bound with greater affinity to the coded particles. Accordingly, the biotinylated cells may be preferentially connected and/or retained on the particles, resulting in enrichment of the transfected cells.

In summary, this technology may be used to attach cells to coded particles. For example, cells are transfected (either stably or transiently) with the aforementioned expression vector. Coded particles are coated with avidin or any of its derivatives. By mixing avidin-coated particles with metabolically biotinylated cells, cell attach to the particles more tightly than through standard cell-substrate interactions. Additionally, in the case of transient transfections, the transfected cells may be separated from the untransfected cells. Typically, if cells are transiently transfected with more than one plasmid, any cell that takes up a single plasmid is likely to take up the others. There are some exceptions to this but, in general, with a three-plasmid scenario, greater than 90% of the transfected cells are assumed to have received all three of the plasmids. Therefore, this technology selects cells that are transfected not only with DNA mediating surface biotinylation, but also by selecting for those transfected with other experimental DNAs (e.g., GPCR or reporter constructs). By doing this, the entire assay platform becomes more robust because 90% of the cells on each particle may be transfected with the appropriate DNA rather than the typical 40-50% obtained by transient transfections in the absence of any selection.

### Example 6

Multiplexed Competitor Screens Using Modified Cell Membranes

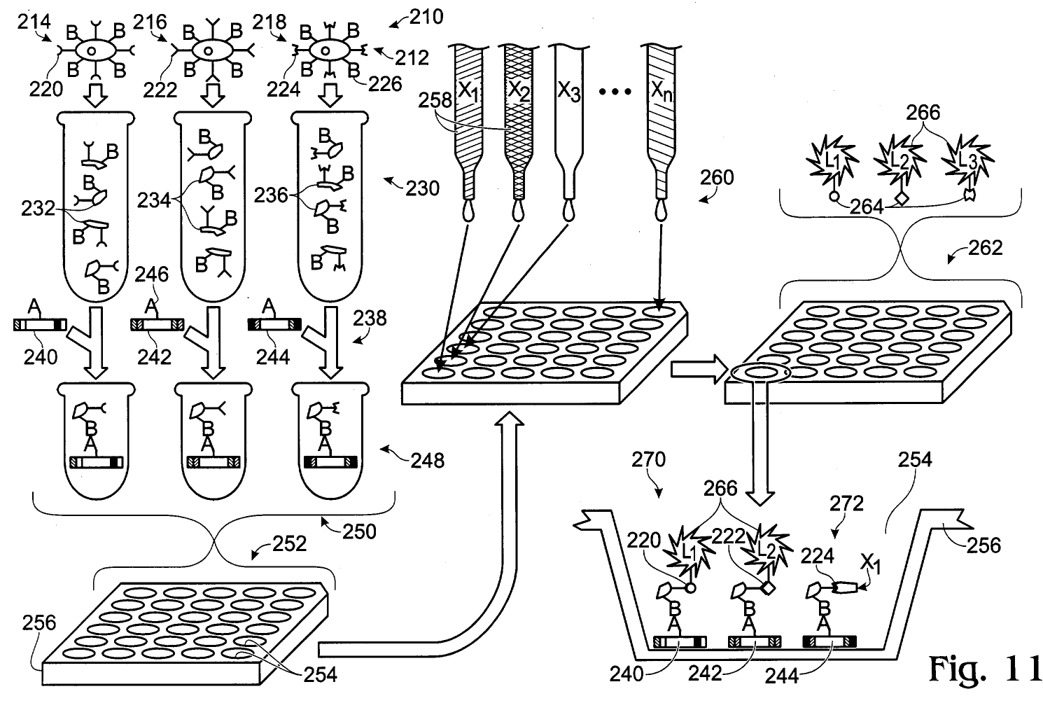

This example describes a method 210 for multiplexed analysis of ligand-receptor interactions by selectively connecting membranes and their receptors to coded particles using association pairs; see FIG. 11.

Method 210 may include providing cells carrying receptors, shown at 212. The cells may include different sets of cells, shown at 214, 216, 218, each expressing different cell-surface receptors 220, 222, 224, respectively. Each different receptor may be selectively expressed by one of the sets of cells, that is, the different receptor is present in greater abundance in the one set than in the other sets. The cells may be different cell types, for example, cells from different tissues and/or lineages that naturally express each different receptor, and/or may be transfected with nucleic acids encoding the different receptors. Each set of cells also may be connected to an association member, such as biotin 226 (“B”). Covalent or noncovalent connection of the association may be conducted as described above in Examples 2 and 5, and in Section IV.

The cells may be fragmented in fluid isolation from one another, shown at 230. Fragmentation may lyse the cells, may release soluble cell contents, and may separate the membranes and their membrane-associated proteins into a plurality of membrane fragments 232, 234, 236, such as membrane vesicles, among others. Fragmentation also may include additional fractionation, such as centrifugation, column chromatography, and/or the like.

Coded particles may be provided, shown at 238. Different classes of coded particles 240, 242, 244 having different codes may be connected to an association partner 246 for association member 226. For example, avidin (“A”) is connected here. Each class of particle then may be contacted with each set of cell fragments 232, 234, 236 to allow association members to be linked covalently or noncovalently. Here, biotinylated fragments bind to avidin on the coated particles, shown at 248. Subsequently, the particles and their linked membrane fragments may be separated from unlinked membranes, soluble cell components, and/or other cell fragments, either by washing the particles or transferring them to another site. As a result of linkage, each different receptor may be identified by a connected code, whereas other cell components and untransfected or unmodified cell membranes may remain unlinked. Thus, in some embodiments, crude cell lysates may be used without a need for fractionation, for example, as may be performed typically by ultracentrifugation.

The fragments then may be mixed and distributed in preparation for ligand binding. Different receptors/membranes and their connected particles may be mixed, shown at 250, to form a mixture in which the different receptors/membranes/particles are randomly positioned relative to one another in a nonpositional array. Portions of the mixture then may be dispensed, shown at 252, to separate compartments, such as wells 254 of a microplate 256. Each portion may include one or more of each type of receptor/membrane, thus providing a positional array of the portions defined by positions of individual wells within the microplate.

The receptors then may be contacted with test compounds and known ligands to assay binding. Receptors may be contacted with a plurality of test compounds 258, shown at 260. A different test compound (or compound mixture), indicated at X₁ to Xₙ, may be added to each well. Before, during, or after addition of test compounds, receptors may be contacted, shown at 264, with a mixture of labeled ligands 264 (indicated as L₁, L₂, and L₃). Each ligand may be configured to bind selectively to one of receptors 220-224, and may include a detectable label 266. For example, ligand L₁ is configured to bind to receptor 220. The same (or a different) label may be connected to each ligand.

Binding may be measured for each well, shown at 270. Binding of labeled ligands 264 to each class of particle may be measured to assay for an ability of each test compound 258 to compete with the labeled ligands for binding to the receptors. In the present illustration, test compound X₁ did not substantially reduce binding of labeled ligands L₁ and L₂ to coded particles 240 and 242 (or receptors 220 and 222), respectively. By contrast, test compound X₁ bound selectively to receptor 224, shown at 272, as indicated by the diminished binding signal from label 266 of ligand L₃ for particles having the code of particle class 244.

### Example 7

Selected Embodiments

This example describes additional selected embodiments of the invention, presented as a series of indexed paragraphs.

1. A method of selectively analyzing a predetermined subcellular derivative, comprising (1) expressing a first member of a specific binding pair on a cellular structure of a cell population; (2) disposing a second member of the specific binding pair on a substrate; (3) lysing the cell population; and (4) exposing the cell population to the substrate after lysing, thereby associating the cellular structure with the substrate through interaction of the first and second members of the specific binding pair.

2. The method of paragraph 1, further comprising the step of measuring a characteristic of the associated cellular structure.

3. The method of paragraph 1, wherein the cellular structure is selected from the group consisting of cell-surface membranes, internal membranes, organelles, nuclei, chromosomes, nuclear matrices, and cytoskeletons.

4. The method of paragraph 1, wherein the step of expressing includes modifying cells of the cell population by introducing at least one nucleic acid molecule that encodes a protein.

5. The method of paragraph 4, the protein being conjugated to the first member posttranslationally.

6. The method of paragraph 5, the protein being a fusion protein having a biotin acceptor site that is a target for biotinylation in the modified cells.

7. The method of paragraph 6, wherein the biotin acceptor site is at least substantially derived from a region of a transcarboxylase from Propionibacterium shermanii.

8. The method of paragraph 4, wherein the at least one nucleic acid molecule includes a regulatory sequence that at least substantially controls expression of the protein, and the regulatory sequence is cell-selective or conditional.

9. The method of paragraph 1, the cellular structure including cell-surface membranes, wherein the step of exposing selectively associates a membrane fraction from the cell population.

10. The method of paragraph 4, wherein the step of modifying introduces at least one nucleic acid molecule encoding a receptor of interest.

11. The method of paragraph 1, wherein the substrate is provided by a carrier selected from the group consisting of microtiter plates, microscope slides, cell culture vessels, test tubes, wafers, planar supports, beads, rods, and microparticles.

12. The method of paragraph 1, the substrate having a nonpartitioned surface, wherein the step of exposing associates the cellular structure with a portion of the surface, thereby enabling the cellular structure from different cell populations to associate with a remaining portion of the surface to form a positional array of cellular structures.

13. The method of paragraph 4, wherein the modified cells are at least substantially non-adherent to the substrate before modifying.

14. The method of paragraph 1, wherein the substrate is a microcarrier, the microcarrier having a detectable code.

15. A method of selectively associating cells with carriers for multiplexed analysis, comprising (1) modifying plural groups of cells to express a first member of a specific binding pair on a cellular structure of the plural groups; (2) disposing a second member of the specific binding pair on a set of carriers, wherein each carrier of the set has a detectable code, and the set includes carriers having distinct codes; and (3) exposing each of the modified plural groups of cells separately to a subset of the carriers having at least one of the distinct codes, so that the cellular structure is associated with the subset through interaction of the first and second members of the specific binding pair, and each of the plural groups is identified by the at least one of the distinct codes.

16. The method of paragraph 15, wherein the set of carriers includes at least three subsets having distinct codes.

17. The method of paragraph 15, wherein the plural groups include at least three groups of cells.

18. The method of paragraph 15, wherein each of the plural groups corresponds to a distinct cell type.

19. The method of paragraph 15, wherein the step of modifying results in expression of a protein of interest, the protein of interest distinguishing each of the plural groups of cells.

20. The method of paragraph 15, the first member comprising biotin, wherein the step of modifying disposes biotin at a cell-surface region of each of the plural groups of cells.

21. A method of forming a composition to assay ligand binding, comprising (1) modifying plural groups of cells to include a first member of a specific binding pair on a cellular structure of the plural groups, each of the plural groups expressing a distinct receptor of interest; (2) disposing a second member of the specific binding pair on a set of carriers, wherein each carrier of the set has a detectable code, and the set includes carriers having distinct codes; (3) lysing each of the plural groups of cells; exposing each of the plural groups of cells separately to a subset of the carriers having at least one of the distinct codes, so that the cellular structure is associated with the subset through interaction of the first and second members of the specific binding pair, and each group is identified by the at least one of the distinct codes; and (4) combining the exposed subsets of carriers to form an arbitrarily distributed array.

22. A composition for multiplexed analysis of cell populations, comprising (1) a set of carriers, each carrier of the set having a code and a first member of a specific binding pair that is attached to the carrier, the code being distinguishable for at least two carriers of the set; and (2) a cell population associated with each carrier of the set, the cell population expressing a second member of the specific binding pair on a surface of the population, wherein the cell population is associated with the carrier through interaction of the first and second members of the specific binding pair, and the code identifies the cell population.

23. The composition of paragraph 22, wherein the cell population includes one or more cells.

24. The composition of paragraph 22, wherein the cell population is modified to express a protein of interest, the protein of interest being identified by the code.

25. The composition of paragraph 22, wherein the set of carriers is associated with plural distinct cell populations, each of the distinct cell populations being associated with one of the distinguishable codes.

26. The composition of paragraph 25, wherein the plural distinct cell populations are modified to express the first member.

27. The composition of paragraph 26, modifying forming the distinct cell population.

28. A kit for analyzing cell populations, comprising a set of carriers, each carrier having a code, and the set including at least two classes of carrier for which the code differs, wherein each carrier includes a first member of a specific binding pair attached the carrier, the first member being capable of specific binding with a second member of the specific binding pair.

29. The kit of paragraph 28, wherein the set includes at least three classes of carriers for which the code differs.

30. The kit of paragraph 28, wherein the specific binding partner is selected from the group consisting of ligands, receptors, antibodies, antigens, biotin, and avidin.

31. The kit of paragraph 28, further including a nucleic acid vector adapted to express a protein, the protein localizing the first member to a cellular structure of cells.

32. The kit of paragraph 31, wherein the protein includes the first member through translation or posttranslational conjugation.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of analyzing cells, comprising:
connecting a first member of an association pair to an external region of the cells;
connecting a second member of the association pair to coded particles of at least two classes, each class having a different code;
joining the first member to the second member to connect the cells to the coded particles; and
performing an assay on at least one of the cells, or a portion thereof, connected to at least one of the coded particles.

2. The method of claim 1, wherein the step of connecting a first member is performed metabolically by the cells.

3. The method of claim 1, wherein the step of connecting a first member connects biotin to the external region, and wherein the step of connecting a second member connects avidin or streptavidin to the coded particles.

4. The method of claim 1, wherein the step of connecting a first member includes introducing a derivative of a cell component into the cells, and metabolically incorporating the derivative into at least one of a glycan and a polypeptide.

5. The method of claim 4, wherein the step of introducing a derivative includes adding the derivative to the fluid medium surrounding the cells, and allowing the derivative to be internalized by the cells.

6. The method of claim 4, wherein the derivative includes the first member or provides a site for conjugation of the first member to the cells.

7. The method of claim 4, wherein the derivative includes at least one of a ketone, a hydrazide, a hydroxylamine, and a thiosemicarbazide.

8. The method of claim 1, wherein the step of connecting a first member includes expressing a polypeptide in the cells by transfection of a nucleic acid encoding the polypeptide.

9. The method of claim 8, wherein the polypeptide includes a site for covalent connection of the first member to the polypeptide after translation of the polypeptide.

10. The method of claim 9, wherein the site is configured to be connected metabolically to the first member, and wherein the first member is biotin.

11. The method of claim 1, wherein the step of connecting a second member results in a covalent connection between the second member and the coded particles.

12. The method of claim 1, wherein the step of connecting a second member results in a noncovalent connection between the second member and the coded particles.

13. The method of claim 1, wherein the step of joining includes forming a covalent bond between the first and second members.

14. The method of claim 1, wherein the first and second members are selected from the group consisting of (1) avidin or streptavidin and biotin, (2) carbohydrate/glycan and lectin or carbohydrate receptor, and (3) antibody and antigen.

15. The method of claim 1, the cells including at least two different sets of cells, wherein the step of joining is performed to connect each different set to a different class of the coded particles.

16. The method of claim 1, further comprising placing one or more coded particles from each of the at least two classes in a plurality of compartments, before the step of performing an assay, and after the step of connecting a second member.

17. The method of claim 1, the cells having a diameter and the coded particles having a length, wherein the diameter is less than the length.

18. The method of claim 1, further comprising the step of fragmenting the cells to form cell fragments, the step of fragmenting being conducted before the step of joining.

19. The method of claim 18, wherein the step of performing an assay is performed on the cell fragments.

20. The method of claim 1, the cells being a subset of a larger set of cells, wherein the steps of connecting a first member and joining are selective for the subset relative to the larger set.

21. The method of claim 1, wherein performing an assay includes performing the assay on at least two of the cells, or portions thereof, connected to coded particles from at least two classes.

22. A method of analyzing cells, comprising:
selecting cells having a first member of an association pair connected to an external region of the cells;
selecting coded particles having a second member of the association pair connected to a region of the coded particles accessible to cells, the coded particles being of at least two classes, each class having a different code;
placing one or more coded particles from each of the at least two classes in a plurality of compartments;
joining the first member to the second member to connect the cells to the coded particles; and
performing an assay on at least one of the cells, or a portion thereof, connected to at least one of the coded particles.

23. The method of claim 22, further comprising modifying the cells so that the first member is covalently connected to the cells in the external region, before the step of selecting cells.

24. The method of claim 23, wherein the step of modifying the cells includes introducing a compound into the cells from a surrounding fluid, the first member being connected to the external region by metabolism of the compound by the cells to covalently connect the first member to the cells.

25. The method of claim 22, further comprising connecting the second member to the coded particles, before the step of selecting coded particles.

26. The method of claim 25, wherein the step of connecting the second member is performed in a separate compartment for each of at the least two classes of coded particles.

27. The method of claim 26, further comprising the step of mixing the at least two classes of coded particles to randomly position the coded particles relative to one another, the step of mixing being performed after the step of connecting the second member.

28. The method of claim 22, wherein the step of joining is conducted before the step of placing.

29. The method of claim 22, wherein the step of joining is performed in a separate compartment for each of the at least two classes of coded particles.

30. A method of analyzing cells, comprising:
fragmenting the cells to produce cell fragments;
connecting a first member of an association pair to the cells or cell fragments;
connecting a second member of the association pair to coded particles of at least two classes, each class having a different code;
joining the first member to the second member to selectively connect a subset of the cell fragments to the coded particles; and
performing an assay on at least one of the cell fragments connected to at least one of the coded particles.

31. The method of claim 30, wherein the subset of cell fragments substantially includes organelles or membranes.

32. The method of claim 30, wherein the step of connecting a first member includes forming a covalent bond between the first member and the cells or cell fragments.

33. The method of claim 30, wherein the cells include a plurality of different sets of cells, each set including a receptor that is present in greater abundance in such set than in the other sets, and wherein joining is conducted to connect the receptor of each set to a different class of the coded particles.

34. The method of claim 30, wherein the step of performing an assay includes measuring binding of a compound to the subset of the cell fragments.

35. A method of analyzing cells, comprising:
connecting a first member of an association pair to a glycan disposed in an external region of the cells;
connecting a second member of the association pair to coded particles of at least two classes, each class having a different code;
joining the first member to the second member to connect the cells to the coded particles; and
performing an assay on at least one of the cells, or a portion thereof, connected to at least one of the coded particles.

36. The method of claim 35, the glycan including a polymer of sugar moieties, wherein the first member is structurally distinct from each of the sugar moieties.

37. The method of claim 35, wherein the step of connecting a first member includes introducing a compound into the cells and metabolically incorporating the compound, or a metabolite thereof, into the glycan.

38. The method of claim 37, wherein the compound or the metabolite includes the first member or provides a site for covalent linkage of the first member to the glycan.

39. A method of preparing cells for multiplexed cellular assays, comprising:
connecting a first member of a chemically reactive pair to cells in an external region of the cells;
providing coded particles of at least two classes, each class having a different code, the coded particles being connected to a second member of the chemically reactive pair; and
forming a covalent bond between the first member and the second member to connect the cells, or fragments thereof, to the coded particles.

40. The method of claim 39, wherein the step of connecting the first member includes metabolically connecting the first member to the cells with a covalent linkage.

41. The method of claim 39, wherein the step of providing coded particles includes providing each class of the coded particles in a separate compartment.

42. The method of claim 39, wherein the step of forming a covalent bond includes reacting a ketone with one of a hydrazide, a hydroxylamine, and a thiosemicarbazide.

43. A kit for connecting cells to particles, comprising:
a reagent configured to covalently connect a first member of an association pair to an external region of cells; and
a set of coded particles of at least two classes, each class having a different code, the coded particles being connected to a second member of the association pair, the second member being configured to be joined to the first member.

44. The kit of claim 43, wherein the reagent includes a saccharide covalently connected to the first member.

45. The kit of claim 43, wherein the reagent is configured to facilitate metabolic connection of the first member to the cells with a covalent linkage.

46. The kit of claim 43, wherein the coded particles in each of the at least two classes is disposed in a separate compartment.
